(19) United States
(12) Patent Application Publication
(10) Pub. No.: US 2026/0108250 A1
(43) Pub. Date: Apr. 23, 2026

(54) RADIOPAQUE SHAPE MEMORY FOAM DEVICES

(71) Applicant: BOSTON SCIENTIFIC SCIMED, INC., Maple Grove, MN (US)

(72) Inventors: Patrick Willoughby, Shoreview, MN (US); Scott Andrew McCullough, Plymouth, MN (US); Rick Dorn, Plymouth, MN (US); Paul Vincent Grosso, Maple Grove, MN (US)

(73) Assignee: BOSTON SCIENTIFIC SCIMED, INC., MAPLE GROVE, MN (US)

(21) Appl. No.: 19/362,860

(22) Filed: Oct. 20, 2025

Related U.S. Application Data
(60) Provisional application No. 63/710,709, filed on Oct. 23, 2024.

(51) Int. Cl. A61B 17/12; A61B 17/00; A61B 90/00; A61L 27/14; A61L 27/50

(52) U.S. Cl. CPC A61B 17/12122; A61B 17/1215; A61L 27/14; A61L 27/50; A61B 2017/00526; A61B 2017/00871; A61B 2090/3966; A61L 2400/16

(57) ABSTRACT

A left atrial appendage closure device may include a shape memory polymer foam and a radiopaque component integrated with the shape memory polymer foam to provide visibility under radiographic based imaging.

### CROSS-REFERENCE TO RELATED APPLICATIONS

This application claims the benefit of priority of U.S. Provisional Application No. 63/710,709 filed Oct. 23, 2024, the entire disclosure of which is hereby incorporated by reference.

### TECHNICAL FIELD

The disclosure relates generally to medical devices and more particularly to medical devices that are adapted for use in percutaneous medical procedures in which an expandable shape memory polymer is inserted into the body.

### BACKGROUND

A wide variety of medical devices have been developed for medical use including, for example, medical devices utilized to occlude regions of the body. These medical devices may be used in a variety of body regions including an aneurysm in a vessel and the left atrial appendage (LAA). In patients suffering from atrial fibrillation, the LAA may not properly contract or empty, causing stagnant blood to pool within its interior, which can lead to the undesirable formation of thrombi within the LAA. Thrombi forming in the LAA may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the LAA. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example left atrial appendage closure device includes a shape memory polymer foam, and a radiopaque component integrated with the shape memory polymer foam to provide visibility under radiographic based imaging.

Alternatively or additionally to the embodiment above, the radiopaque component is incorporated into a chemical structure of the shape memory polymer foam.

Alternatively or additionally to any of the embodiments above, the radiopaque component is coated on a surface of the shape memory polymer foam.

Alternatively or additionally to any of the embodiments above, the radiopaque component is spray coated or dip coated onto the shape memory polymer foam.

Alternatively or additionally to any of the embodiments above, the shape memory polymer foam comprises a pocket containing the radiopaque component.

Alternatively or additionally to any of the embodiments above, the pocket is configured to release the radiopaque component when the shape memory polymer foam expands.

Alternatively or additionally to any of the embodiments above, the radiopaque component is trapped within pores of the shape memory polymer foam.

Alternatively or additionally to any of the embodiments above, the radiopaque component comprises a contrast agent selected from the group consisting of iodixanol, iohexol, and iopromide.

Alternatively or additionally to any of the embodiments above, the shape memory polymer foam comprises an isocyanate-based polyurethane foam.

An example method of manufacturing a left atrial appendage closure device includes forming a shape memory polymer foam, and integrating a radiopaque component with the shape memory polymer foam to provide visibility under radiographic based imaging.

Alternatively or additionally to any of the embodiments above, integrating the radiopaque component includes incorporating the radiopaque component into a chemical structure of the shape memory polymer foam during synthesis.

Alternatively or additionally to any of the embodiments above, incorporating the radiopaque component includes using a polyol or ionic iodinated component in the synthesis of the shape memory polymer foam.

Alternatively or additionally to any of the embodiments above, integrating the radiopaque component includes coating a surface of the shape memory polymer foam with a contrast agent.

Alternatively or additionally to any of the embodiments above, coating the surface includes spray coating or dip coating the contrast agent onto the shape memory polymer foam.

Alternatively or additionally to any of the embodiments above, the method further includes crimping the shape memory polymer foam to create a pocket, and injecting the radiopaque component into the pocket.

Alternatively or additionally to any of the embodiments above, the method further includes absorbing a contrast solution into the shape memory polymer foam and crimping the shape memory polymer foam to trap the contrast solution within pores of the shape memory polymer foam.

Alternatively or additionally to any of the embodiments above, integrating the radiopaque component includes incorporating a polyol or ionic iodinated component into the shape memory polymer foam during synthesis.

Alternatively or additionally to any of the embodiments above, the method further includes applying a dissolvable adhesive to temporarily adhere the radiopaque component to the shape memory polymer foam.

Another example left atrial appendage closure device includes an expandable frame, and a shape memory polymer foam disposed within the expandable frame, the shape memory polymer foam having a radiopaque component integrated therewith to provide visibility under radiographic based imaging, where the shape memory polymer foam is configured to expand when exposed to body temperature to fill at least a portion of the expandable frame.

Alternatively or additionally to any of the embodiments above, the radiopaque component comprises a contrast agent selected from the group consisting of iodixanol, iohexol, and iopromide.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a prior art expandable LAA implant;

FIGS. 2A and 2B illustrate foam implants under radiographic (X-ray) based imaging;

FIGS. 3A, 3B, 4, and 5 are cross-sectional views of example shape memory polymer (SMP) foam implants with a radiopaque component; and

FIG. 6 is a cross-sectional view of an example expandable frame with a radiopaque foam implant disposed therein.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

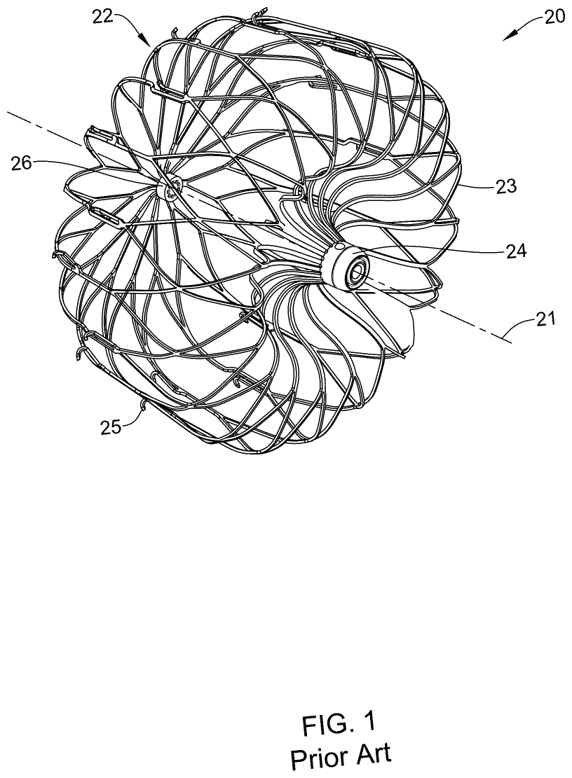

Various medical devices have been developed which are deployed to close off the left atrial appendage (LAA). FIG. 1. illustrates selected components of a prior art occlusive implant 20 for occluding the LAA. The occlusive implant 20 includes an expandable framework 22 configured to shift along a longitudinal axis 21 between the collapsed configuration and the deployed configuration. The expandable framework 22 may comprise a plurality of interconnected struts 23 defining a plurality of cells. The plurality of cells may be a plurality of closed cells, open cells, or the plurality of cells may include a plurality of open cells and a plurality of closed cells in various combinations and/or arrangements. The occlusive implant 20 and/or the expandable framework 22 may comprise a plurality of anchoring elements 25. In some embodiments, the plurality of anchoring elements 25 may extend radially outward from the expandable framework 22 in the deployed configuration. The plurality of anchoring elements 25 may be configured to engage with tissue and/or may be configured to secure the occlusive implant 20 and/or the expandable framework 22 to tissue at a target site (e.g., the left atrial appendage, etc.). The plurality of anchoring elements 25 may be configured to prevent dislodgement and/or ejection of the occlusive implant 20 from the target site. The occlusive implant 20 and/or the expandable framework 22 may include a proximal hub 24 and a distal hub 26. The longitudinal axis 21 of the expandable framework 22 may extend from the proximal hub 24 to the distal hub 26. The plurality of interconnected struts may be joined together at and/or fixedly attached to the proximal hub 24 and/or the distal hub 26. The expandable framework 22, anchoring elements 25, and/or other components of the occlusive implant 20 may be formed from shape memory materials such as nitinol.

Alternatively, or in addition to an expandable implant 20 such as that shown in FIG. 1, expandable foams may be utilized for occluding the LAA. Shape memory polymers (SMPs) are a class of materials which can have multiple geometric and mechanical configurations when exposed to temperature and chemical environments. These polymers can be synthesized into foam structures with tunable thermomechanical properties and foam architectures. SMP foams offer advantages over traditional shape memory materials such as nitinol, including lower stiffness, larger deformation capabilities which are generally not reversible in-use, and can be tailored to provide different properties at a low cost. The synthesis of SMP foams typically involves the reaction of isocyanates or diisocyanates with polyols, catalysts, and surfactants.

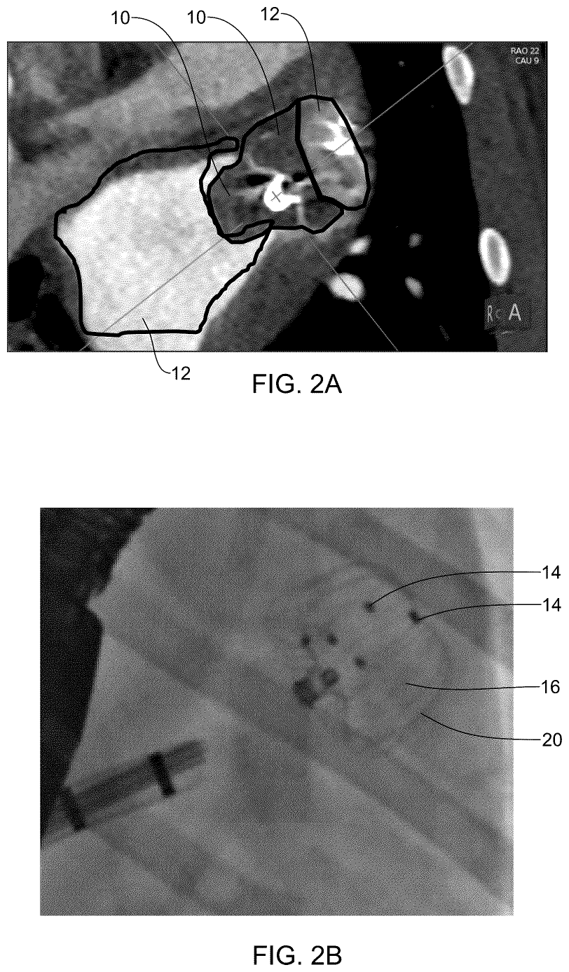

When SMP foams are used in some medical procedures, including implants for occluding the LAA, aspects of or the overall SMP foam structure might not appear on radiographic (X-ray) based imaging without additional influence. As an example, one method of visualizing an SMP foam on an X-ray image involves injecting a contrast dye or agent during a delivery, surveying, or potential readjustment medical procedure. FIG. 2A is a radiographic (X-ray) based image of a plurality of conventional SMP foam structures 10 disposed within the body, with regions of injected contrast agent 12 showing on the periphery. This method does not provide visualization of the SMP foam implant itself and is often time limited as the injected contrast agent may be washed away, particularly when the implant is readjusted. Another way to make an SMP foam structure visible on X-ray is to couple metallic radiopaque markers to the SMP foam structure. FIG. 2B is a radiographic (X-ray) based image of an LAA occlusive implant 20 with an SMP foam structure 16 carrying a plurality of radiopaque markers 14 disposed inside the occlusive implant 20. This method has a drawback of providing limited visibility only on the areas that carry the markers, and the markers may interfere with compression and expansion of the foam implant.

Incorporating a radiopaque component into the chemical backbone of the shape memory polymer foam offers an advantage of potentially providing more uniform radiopacity throughout the entire structure. This approach may result in better overall visibility under radiographic (X-ray) based imaging. However, it may have the significant drawback of potentially interfering with the foam's expansion properties and other secondary actions that enable self-expansion. To address this limitation, various techniques have been developed to form a medical device including an SMP foam and a radiopaque component integrated into the chemical or physical structure of the SMP foam medical device to provide visibility under radiographic (X-ray) based imaging, while minimizing impact on their performance. These techniques include disposing a radiopaque component on the surface of an SMP foam, which may allow for maintaining the desired expansion characteristics of the foam while still providing visibility of the entire SMP foam implant under radiographic (X-ray) based imaging. Surface adhesion of the radiopaque component may offer more flexibility in terms of application methods, such as spray coating or dip coating, and allows for the use of various contrast agents or radiopaque solids. Another method may involve creating contrast-filled pockets within the foam, or trapping contrast solutions within the foam's pores. Additionally, incorporating radiopaque chemistry into the foam's structure may be used.

In the following described embodiments, the radiopaque component that may be coated on the surface, secured within the pores, or incorporated into the material composition of the SMP foam are identified in Table 1. Polyol iodinated compounds contain two or more alcohol or hydroxide functional groups. The alcohol or hydroxide functional group can serve as a hydrogen bonding donor and acceptor reactive sites that may enable hydrogen bonding with isocyanate-based chemistries to assemble polyurethane foams. Ionic iodinated compounds contain carboxylates/carboxylic acid functional groups that provide the reactive sites to enable bonding with isocyanate-based chemistries. Alternatively or additionally, the radiopaque component may include one or more suspended radiopaque solids such as tantalum and zinc oxide.

TABLE 1
Aromatic Iodinated Contrast Agents.

| Compound Name | Type | CAS Number |
|---|---|---|
| Iopromide | Polyol | 73334-07-3 |
| Metrizamide | Polyol | 31112-62-6 |
| Iopamidol | Polyol | 60166-93-0 |
| Iomeprol | Polyol | 78649-41-9 |
| Ioxilan | Polyol | 107793-72-6 |
| Iopentol | Polyol | 89797-00-2 |
| Ioversol | Polyol | 87771-40-2 |
| Iohexol | Polyol | 66108-95-0 |
| Iobitridol | Polyol | 136949-58-1 |
| Iodixanol | Polyol | 92339-11-2 |
| Diatrizoate | Ionic | 117-96-4 |
| Metrizoate | Ionic | 1949-45-7 |
| Ioxitalamic acid | Ionic | 28179-44-4 |
| Ioseric acid | Ionic | 51876-99-4 |
| Iothalamic acid | Ionic | 2276-90-6 |
| Ioglicic acid | Ionic | 49755-67-1 |
| Ioxaglic acid | Ionic | 59017-64-0 |
| Ioglycamic acid | Ionic | 2618-25-9 |
| Iocarmic acid | Ionic | 10397-75-8 |

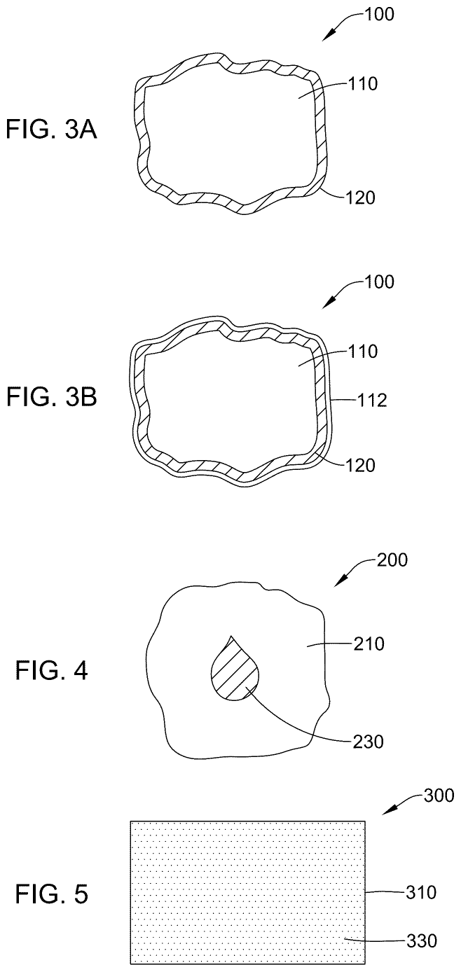

In one embodiment, an SMP foam implant 100 may be formed by applying a coating 120 of one or more radiopaque components onto the outer surface of an SMP foam element 110, as shown in FIGS. 3A and 3B. For example, the radiopaque component may be formed as a solution and spray coated or dip coated onto the SMP foam element 110, either in its expanded configuration or after it has been crimped for delivery. The expanded SMP foam element 110 may be dipped or sprayed with a dilute solution of radiopaque component to fully coat all pores. Alternatively, the SMP foam element 110 may first be crimped and then dipped or sprayed with the dilute solution of radiopaque component to coat only the outer pores that remain exposed after crimping. In other embodiments, the solution of radiopaque component may be utilized as a flushing solution during device preparation, being drawn into the SMP foam during initial expansion in the delivery catheter. The radiopaque component may be coated on the radially outermost surface along a length of the SMP foam element 110. In other embodiments, the coating 120 may be positioned only on the proximal and distal faces of the SMP foam element 110, on the entirety of the exterior surface, or locally as one or more marker band strips, local spots, etc.

Upon delivery into the body and expansion of the SMP foam element or implant 100, the radiopaque coating 120 may provide for visualization of the entire outer extent of the SMP foam implant 100 on a radiographic (X-ray) based image. The radiopaque coating 120 may be configured to be permanently adhered to the outer surface of the SMP foam element 110. In some embodiments, an adhesive may be applied with or over the radiopaque coating 120 to adhere the radiopaque component. The adhesive may be dissolvable, allowing the radiopaque coating 120 to dissipate as the implanted SMP foam implant 100 is exposed to body fluids. In another embodiment, a portion of or the entire surface of the SMP foam implant 100 may be coated with a gel coating containing the radiopaque coating 120, where the gel coating is configured to slowly dissolve after implantation. In other embodiments, a dissolvable or permanent adhesive 112 may be applied with the radiopaque coating 120 or over the coating 120 in order to maintain the radiopaque coating 120 on the SMP foam element 110 for a longer period of time. In other embodiments, a moisture barrier 112 may be coated over the radiopaque coating 120 to retain the coating 120 for a longer time period as compared to without the moisture barrier.

In some embodiments, the dissolvable or permanent adhesive or moisture barrier 112 may be applied either in the expanded configuration or the compressed configuration of the SMP foam element 110. The adhesive or barrier 112 may be applied by dipping the SMP foam element 110 into a solution of the adhesive. A solution of the adhesive or barrier 112 may be sprayed or painted onto the SMP foam element 110, for example. In some cases, the adhesive or barrier may be an integral part of the polymeric makeup of the shape radiopaque coating 120.

A variety of different materials may be used as, or included within, the adhesive or barrier 112. In some cases, the adhesive or barrier 112 may include fibrin. The adhesive or barrier 112 may include a polyurethane or cyano-acrylate material.

In some embodiments, the radiopaque component may be configured to be soluble during implantation and final positioning, gradually being released from the surface of the SMP foam implant as the SMP foam implant pores are washed distally with flushing fluid, blood or other body fluid during the procedure. In this way, the radiopaque coating 120 may be used for procedural assessment of how well the SMP foam implant 100 seals the body lumen in which it is deployed, such as the LAA. In some embodiments, the radiopaque coating solution may contain suspended radiopaque solids that are transported into the interior of the SMP foam through solvent, and then deposited on the surface when dried. The radiopaque component may be coated in a concentration and/or thickness to provide a desired time period of X-ray visibility, such as for delivery and final positioning. In some embodiments, the radiopaque component(s) may be present in a concentration of between 1% to 25% of the total SMP foam element composition, for SMP foam elements of between 10 mm to 50 mm.

The radiopaque component may be configured to provide radiographic (X-ray) based visualization over a range of time periods, depending on the situation. In some embodiments, the radiopaque component may provide radiographic (X-ray) based imaging for up to 2-5 minutes when the radiopaque coating is also used to change the rate of foam expansion. In other embodiments, the radiopaque component may provide radiographic (X-ray) based imaging for up to 10 minutes during the implant process to watch how the foam expands. In further embodiments, the radiopaque component may provide radiographic (X-ray) based imaging for up to 8 hours to monitor the implant during the sub-acute window, for example when the patient is on the table or in the hospital for follow-up due to complications. Additionally, the radiopaque component may provide radiographic (X-ray) based imaging for between 30-45 days to monitor the implant during chronic healing for drug indications. The length of time for which the radiopaque component provides radiographic (X-ray) based imaging may depend on one or more of the concentrations and/or type of radiopaque material, thickness, and position on or in the left atrial appendage closure implant.

The bulk of the coated SMP foam implant 100 would be opaque under radiographic (X-ray) based imaging and would appear darker in the crimped state during delivery, and brighter and easily visualized in the expanded state within the body.

In another embodiment, an SMP foam implant 200 may be formed with at least one inner pocket 230 within an SMP foam element 210, as shown in FIG. 4. The inner pocket 230 may then be filled with one or more radiopaque component. The inner pocket 230 may be formed by crimping the SMP foam element 210 around one or more mandrels to create one or more inner pockets 230. After crimping, a contrast solution including one or more radiopaque components may be absorbed into the SMP foam or injected directly into the inner pocket 230. In other embodiments, a plurality of holes may be drilled radially into the formed SMP foam implant 200, before or after crimping the implant for delivery. The radiopaque component may be injected into the pocket(s) or holes. The pocket or holes may be configured to release the radiopaque component when the SMP foam implant 200 expands in the body.

In a further embodiment, an SMP foam implant 300 may be formed by trapping one or more radiopaque components 330 within the pores of an SMP foam element 310, as shown in FIG. 5. For example, the SMP foam element 310 may be spray or dip coated with a solution of radiopaque component 330 and then when the SMP foam element 310 has absorbed the radiopaque component, the SMP foam element 310 may be crimped, resulting in the solution of radiopaque component 330 being trapped within the pores of the crimped SMP foam element 310. After implantation in the body, the SMP foam implant 300 will expand in the presence of body fluids, which may eventually expose and release the radiopaque component. Trapping the radiopaque component 330 within the pores of the crimped SMP foam implant 300 will provide sufficient radiopacity for visualizing the SMP foam implant 300 during delivery and final positioning.

In another embodiment, a medical device is provided that includes an SMP foam and a radiopaque component incorporated into the chemical structure of the foam during synthesis to provide visibility under radiographic (X-ray) based imaging. The radiopaque component may include a contrast agent, such as, but not limited to, the compounds identified in Table 1. A method of manufacturing a radiopaque shape memory foam device is also provided. The method comprises forming a shape memory polymer foam and incorporating the radiopaque component into the chemical structure of the foam during synthesis, such as using an iodinated contrast agent that is incorporated into final polyurethane foam. In some embodiments, polyol or ionic iodinated components may be used in a foaming process to incorporate iodinated species into the SMP foam. For example, the polyol or ionic iodinated components may be added at the end of the foaming process to primarily coat the surface or be injected into the pores of the resulting SMP foam to maintain the desired foam properties.

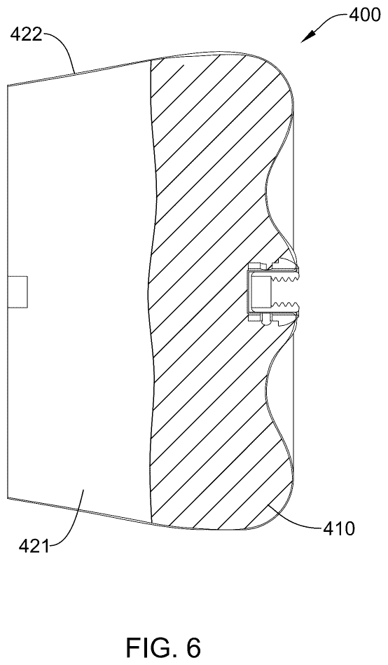

In a further embodiment, a left atrial appendage closure device 400 is provided, including an expandable frame 422 and a radiopaque SMP foam element 410 is disposed within the frame, as shown in FIG. 6. The expandable frame 422 may define an interior 421 space and may be the same as the expandable framework 22 shown in FIG. 1. The SMP foam element 410 may be formed as described in any of the above embodiments, having a radiopaque component integrated therewith to provide visibility under radiographic (X-ray) based imaging. The radiopaque component may be one or more of a contrast agent incorporated into a chemical structure of the shape memory polymer foam, a contrast agent coated on a surface of the shape memory polymer foam, a contrast agent contained within a pocket in the shape memory polymer foam, and a contrast agent trapped within pores of the shape memory polymer foam. The SMP foam element 410 is configured to expand when exposed to body fluids to fill at least a portion of the expandable frame 422.

In some embodiments, in the expanded configuration, the SMP foam element 410 may be configured to fill at least 40% of the interior 421 of the expandable frame 422 in the deployed configuration. In other embodiments, in the expanded configuration, the SMP foam element 410 may be configured to fill at least 80%, at least 85%, at least 90%, or at least 95% of the interior 421 of the expandable frame 422 in the deployed configuration.

In some embodiments, the SMP foam element 410 may be configured to remain within the interior 421 of the expandable frame 422 permanently (e.g., the SMP foam element 410 is never removed from the interior 421 of the expandable frame 422 by a practitioner). In some embodiments, the SMP foam element 410 may be configured to be biodegradable over time. In some embodiments, the SMP foam element 410 may be configured to be biodegradable over at least 30 days. In some embodiments, the SMP foam element 410 may be configured to be biodegradable over at least 60 days. In some embodiments, the SMP foam element 410 may be configured to be biodegradable over at least 90 days. In some embodiments, the SMP foam element 410 may be configured to be biodegradable over at least 180 days. In some embodiments, the SMP foam element 410 may be configured to be biodegradable over at least 365 days. Other configurations are also contemplated.

The chemical structure of the radiopaque shape memory polymer foam can be modified to control its expansion temperature or mechanical properties. This can be achieved through various methods. In one method, the ratio of hard segments (isocyanates) to soft segments (polyols) may be adjusted to tune the glass transition temperature and mechanical properties of the foam. In another method, different types of isocyanates may be used, such as, but not limited to, hexamethylene diisocyanate (HDI), isophorone diisocyanate, or trimethyl hexamethylene diisocyanate (TMHDI), in order to affect the foam's properties. Another method may involve modifying the types or ratios of polyol or ionic iodinated components used in making the SMP foam, in order to influence the foam's characteristics. Another method involves integrating polyol or ionic iodinated components into the foam synthesis to not only provide radiopacity but also to affect the foam's properties. A further method may involve adjusting catalyst and surfactant concentrations used in the foam synthesis.

Catalysts and surfactants play crucial roles in the formation and final properties of shape memory polymer foams. Adjusting their concentrations can affect several aspects of the foam. For example, changing the concentrations of catalysts and surfactants can influence the foam structure including the size, distribution, and interconnectivity of pores within the foam. This, in turn, affects the foam's overall structure and mechanical properties. The reaction kinetics may also be altered. Catalysts can alter the rate of polymerization and cross-linking reactions during foam formation. By adjusting catalyst concentrations, the speed of foam formation and the degree of cross-linking can be controlled, which impacts the foam's final properties. Surface properties may also be altered. Surfactants affect the surface tension and interfacial properties during foam formation. Modifying surfactant concentrations can influence the foam's surface characteristics, potentially affecting how well radiopaque components adhere to or integrate with the foam surface. The Expansion behavior may be altered. The concentrations of catalysts and surfactants can impact the foam's expansion behavior, including its rate of expansion and final expanded state. This is particularly important for shape memory foams used in medical devices where controlled expansion is crucial. Finally, the mechanical properties may be altered. By influencing the foam's structure and cross-linking density, changes in catalyst and surfactant concentrations can affect the foam's mechanical properties such as stiffness, elasticity, and shape memory behavior.

The above described radiopaque SMP foam devices may offer several advantages, such as visibility under radiographic (X-ray) based imaging without the need for separate radiopaque markers or contrast injection during procedures, customizable mechanical properties and expansion temperatures, and the potential for use in various medical devices, particularly in left atrial appendage closure devices.

In some embodiments, the radiopaque SMP foam implant may be configured to prevent thrombus formation (e.g., within the left atrial appendage). In some embodiments, the radiopaque SMP foam implant may include anti-thrombus medicament(s). In some embodiments, the radiopaque SMP foam implant may be configured to absorb blood and/or bodily fluid(s). In some embodiments, the radiopaque SMP foam implant may be configured to trap thrombus. In some embodiments, the radiopaque SMP foam implant may be configured to promote tissue ingrowth and/or endothelization. Other configurations are also contemplated.

In at least some embodiments, the radiopaque SMP foam implant may be configured as open celled foam. The SMP foam may have multiple geometric and/or mechanical properties when exposed to temperature, moisture, and/or chemical environments, and/or changes therein. In some embodiments, the SMP foam may have a collapsibility ratio that is high. The collapsibility ratio is a ratio between an expanded size and a collapsed size. In some examples, the collapsibility ratio of the SMP foam may be at least 5 times, at least 7 times, at least 8 times, at least 9 times, at least 10 times, at least 12 times, or more.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the expandable frame.

The SMP foam may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. Suitable transition temperatures may be, for example, at or below about 37° C. (about 98.6° F.), which allows the shape memory foam to assume an initial configuration prior to and during delivery through a delivery catheter or other delivery device, and an expanded configuration for occlusion after delivery and release within the left atrial appendage, allowing the shape memory foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from about 10 kg/m3 (about 0.62 lb/ft3) to about 1000 kg/m3 (about 62.31 lb/ft3), including from about 10 kg/m3 to about 500 kg/m3 (about 31.2 lb/ft3) including from about 10 kg/m3 to about 200 kg/m3 (about 12.5 lb/ft3), including from about 20 kg/m3 to about 100 kg/m3 (about 6.2 lb/ft3).

Generally, the material for constructing the SMP foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the SMP foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the SMP foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D,L-lactide, poly(beta-hydroxybutyrate), polygluconate including poly-D-gluconate, poly-L-gluconate, poly-D,L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly [1,3bis(p-carboxyphenoxy) propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerzation of various diketene acetals and diols; (d) polyanhydride homopolymers and copolymers such as poly(adipic anhydride), poly(suberic anhydride), poly(sebacic anhydride), poly(dodecanedioic anhydride), poly(maleic anhydride), poly [1,3-bis-(p-carboxyphenoxy) methane anhydride], and poly [alpha, omega-bis(p-carboxyphenoxy) alkane anhydride] such as poly [1,3-bis(p-carboxyphenoxy) propane anhydride] and poly [1,3-bis(p-carboxyphenoxy) hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the SMP foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others. In some cases, the shape memory polymers may include non-foamed versions of the polymers described herein with respect to making the expandable foams such as shape memory foams. Example of bio-compatible shape memory polymers include polymers made from poly(ε-caprolactone) (PCL), polyurethane (PU), poly (D, L-lactide) (PDLLA), PVA, ethylene vinyl acetate copolymer, (EVA) polymer blend, polymer composites, crosslinked polymers and supramolecular networks, among others. In some instances, shape memory polymers that may be used in creating the foamable solutions described herein may include polyurethane, for example.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, the expandable frame may include a fabric material disposed on, for example, the proximal end. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A left atrial appendage closure device comprising:
a shape memory polymer foam; and
a radiopaque component integrated with the shape memory polymer foam to provide visibility under radiographic based imaging.

2. The left atrial appendage closure device of claim 1, wherein the radiopaque component is incorporated into a chemical structure of the shape memory polymer foam.

3. The left atrial appendage closure device of claim 1, wherein the radiopaque component is coated on a surface of the shape memory polymer foam.

4. The left atrial appendage closure device of claim 3, wherein the radiopaque component is spray coated or dip coated onto the shape memory polymer foam.

5. The left atrial appendage closure device of claim 1, wherein the shape memory polymer foam comprises a pocket containing the radiopaque component.

6. The left atrial appendage closure device of claim 5, wherein the pocket is configured to release the radiopaque component when the shape memory polymer foam expands.

7. The left atrial appendage closure device of claim 1, wherein the radiopaque component is trapped within pores of the shape memory polymer foam.

8. The left atrial appendage closure device of claim 1, wherein the radiopaque component comprises a contrast agent selected from the group consisting of iodixanol, iohexol, and iopromide.

9. The left atrial appendage closure device of claim 1, wherein the shape memory polymer foam comprises an isocyanate-based polyurethane foam.

10. A method of manufacturing a left atrial appendage closure device, the method comprising:
forming a shape memory polymer foam; and
integrating a radiopaque component with the shape memory polymer foam to provide visibility under radiographic based imaging.

11. The method of claim 10, wherein integrating the radiopaque component comprises incorporating the radiopaque component into a chemical structure of the shape memory polymer foam during synthesis.

12. The method of claim 11, wherein incorporating the radiopaque component comprises using a polyol or ionic iodinated component in the synthesis of the shape memory polymer foam.

13. The method of claim 10, wherein integrating the radiopaque component comprises coating a surface of the shape memory polymer foam with a contrast agent.

14. The method of claim 13, wherein coating the surface comprises spray coating or dip coating the contrast agent onto the shape memory polymer foam.

15. The method of claim 10, further comprising crimping the shape memory polymer foam to create a pocket, and injecting the radiopaque component into the pocket.

16. The method of claim 10, further comprising absorbing a contrast solution into the shape memory polymer foam and crimping the shape memory polymer foam to trap the contrast solution within pores of the shape memory polymer foam.

17. The method of claim 10, wherein integrating the radiopaque component comprises incorporating a polyol or ionic iodinated component into the shape memory polymer foam during synthesis.

18. The method of claim 10, further comprising applying a dissolvable adhesive to temporarily adhere the radiopaque component to the shape memory polymer foam.

19. A left atrial appendage closure device comprising:
an expandable frame; and
a shape memory polymer foam disposed within the expandable frame, the shape memory polymer foam having a radiopaque component integrated therewith to provide visibility under radiographic based imaging;
wherein the shape memory polymer foam is configured to expand when exposed to body temperature to fill at least a portion of the expandable frame.

20. The left atrial appendage closure device of claim 19, wherein the radiopaque component comprises a contrast agent selected from the group consisting of iodixanol, iohexol, and iopromide.
